(19) United States
(12) Patent Application Publication
(10) Pub. No.: US 2021/0186534 A1
(43) Pub. Date: Jun. 24, 2021

(54) ASPIRATION CATHETER AND PUMP SYSTEM FOR TREATING ISCHEMIC STROKE

(71) Applicant: POSEYDON MEDICAL, LLC, Mirimar, FL (US)

(72) Inventors: TIMOTHY BRANDON HUNT, Miramar, FL (US); ROBERT FARNAN, Miramar, FL (US); DUANE COURI, Miramar, FL (US); ALEJANDRO ESPINOSA, Miramar, FL (US)

(73) Assignee: POSEYDON MEDICAL, LLC, MIRIMAR, FL (US)

(21) Appl. No.: 17/075,805

(22) Filed: Oct. 21, 2020

Related U.S. Application Data
(60) Provisional application No. 62/949,477, filed on Dec. 18, 2019.

(51) Int. Cl. A61B 17/22

(52) U.S. Cl. CPC A61B 17/22; A61B 2017/22045; A61B 2017/00199; A61B 2017/22084; A61B 2017/22079

(57) ABSTRACT

The present invention relates to methods and systems for performing the removal of thrombus from a vessel lumen. More particularly the present invention relates to a thrombectomy system that includes an elongate catheter and a disposable aspiration pump and methods of performing medical procedures to remove clots, thrombus and emboli to re-establish the normal intravascular flow of blood.

### CROSS REFERENCE TO RELATED APPLICATION

This application claims priority to U.S. Provisional Application Ser. 62/949,477, filed Dec. 18, 2019, the disclosure of which is incorporated by reference in its entirety.

### BACKGROUND OF THE INVENTION

For many years catheters have been used to within the vasculature for diagnostics and therapeutic purposes. These therapies include treatments for ischemia in which removal of an occlusion is performed in the vasculature to re-establish normal blood flow. The blockage may be due to thrombus, plaque, foreign objects or a combination thereof. Generally, soft thrombus created elsewhere in the body (for example due to atrial fibrillation) that lodges in the distal cerebrovasculature may be disrupted or dissolved using mechanical devices and or thrombolytic drugs. When treating ischemia in cerebral vessels, small diameter, flexible microcatheters are typically used because they can navigate the tortuous anatomy to access the site of the occlusion. These small diameter microcatheters typically have outer diameters from 1.0 to 2.0 millimeters, inner diameters of 0.5 to 1.5 mm like those described in U.S. Pat. No. 6,197,014 to Samson et al., entitled, “Kink-resistant braided catheter with distal side holes” and are used to deliver therapeutic materials such as clot dissolving drugs, mechanical thrombus retrieval or disruption devices. While guidewires are typically used to disrupt the thrombus, some sophisticated thrombectomy devices have been proposed. For instance U.S. Pat. No. 4,762,130 to Fogarty et al., entitled, “Catheter with Corkscrew-Like Balloon”, U.S. Pat. No. 4,998,919 of Schepp-Pesh et al., entitled, “Thrombectomy Apparatus”, U.S. Pat. No. 5,417,703 to Brown et al., entitled “Thrombectomy Devices and Methods of Using Same”, and U.S. Pat. No. 6,663,650 to Sepetka et al., entitled, “Systems, Methods and Devices for Removing Obstructions from a Blood Vessel” discloses devices such as catheter based corkscrew balloons, baskets or filter wires and helical coiled retrievers. Commercial and prototype versions of these devices have shown only marginal improvements over guidewires due to an inability to adequately grasp the thrombus or to gain vascular access distal to the thrombus (i.e. distal advancement of the device pushes the thrombus distally).

Aspiration or suction may be applied to the catheter lumen to aid in removing the thrombus from the occlusion site. Due to the size of the catheter inner diameter the thrombus is typically broken into smaller pieces to facilitate removal through aspiration. If the thrombus includes organized tissue it may be unable to be broken into small pieces and the larger pieces may become lodged in the microcatheter inner diameter requiring that the entire microcatheter be removed to remove the blockage. Should this occur, valuable time to treat the patient is wasted and may lead to a poor outcome for the patient. There is a need for thrombectomy system that incorporates an optimized catheter having a large lumen and an aspiration pump to rapidly remove thrombus including organized thrombus without fragmenting the thrombus.

### SUMMARY

In accordance with one aspect there is provided a medical device system for restoring patency of a body lumen in a mammal. More particularly, there is provided a thrombectomy system which includes an elongate thrombectomy catheter having a proximal end with a hub assembly and a distal end, with proximal, intermediate and distal sections positioned between the proximal end and distal end and an aspiration pump that can be coupled to the catheter proximal end. The elongate catheter is constructed of different polymers having various durometers and includes reinforcement materials to provide a catheter lumen having a large inner diameter, greater than 0.085 inches, and whose distal section can be subjected to a bend radius of two times the inner diameter without kinking. The elongate catheter is has a distal section that preferably includes a helical wire reinforcement and is of a construction that can be subjected to negative pressures of 29 inHg without causing catastrophic damage that would render the catheter unusable.

In accordance with another aspect of the present invention there is provided a thrombectomy system catheter assembly comprising biocompatible resilient materials. Suitable resilient materials include metal alloys such as nitinol, titanium, stainless steel and cobalt chromium and any alloys thereof. Additional suitable materials include polymers such as polyimides, polyamides, fluoropolymers, polyetheretherketone (PEEK), polyurethanes, EPTFE, polyesters and shape memory polymers. These materials may be formed into desired shapes by a variety of methods which are appropriate to the materials being utilized such as extrusion, laser cutting, injection molding, welding, electrochemical machining, machining, photo-etching and casting.

In accordance with yet another aspect there is provided an aspiration pump that is compact and disposable. The aspiration pump includes a housing that contains the pump assembly, a removable aspiration container, a power module and a programmable controller module. The aspiration pump includes other modules/components such as a sensor module, an audio module, a display module, a data storage module and an input output module whereby the programmable controller can wirelessly receive or send programs or data to or from external devices.

In accordance with another aspect there is provided a method for performing a thrombectomy procedure using a thrombectomy system that includes an elongate catheter having proximal and distal ends and an inner diameter greater than 0.085 in diameter, an aspiration pump and a connector coupling the catheter and pump. The method includes the steps of:
providing an elongate catheter having proximal and distal ends and an inner diameter greater than 0.085 in;
providing an aspiration pump having an aspiration container;
positioning the distal end of the catheter within a vessel lumen wherein the inner diameter of the catheter distal end is greater than 50% of the inner diameter of the vessel lumen and adjacent a thrombus;
coupling the aspiration pump to the catheter using a connector;
operating the aspiration pump to provide negative pressure to the lumen of the catheter thereby suctioning thrombus through the catheter lumen and into the aspiration container;
withdrawing the catheter from the vasculature.

### BRIEF DESCRIPTION OF THE DRAWINGS

FIG. 1 is a partial cross-sectional view of a thrombectomy system for removing thrombus in a targeted area of the vasculature including a catheter and aspiration pump.

FIG. 2 is an enlarged partial cross-sectional view of the distal end of the catheter.

FIG. 3 is an enlarged partial cross-sectional view of the distal end of the catheter placed in a bent configuration.

FIG. 4A-4C are respectively a side view of the aspiration pump, a schematic of the pump functional modules and a top view of the aspiration pump of the thrombectomy system of FIG. 1.

FIG. 5 is a partial cross sectional view of the distal end of the catheter, shown in FIG. 1, positioned within the lumen of a vessel adjacent a thrombus.

FIG. 6 is a method of removing a thrombus from a vessel using a thrombectomy system shown in FIG. 1.

### DETAILED DESCRIPTION

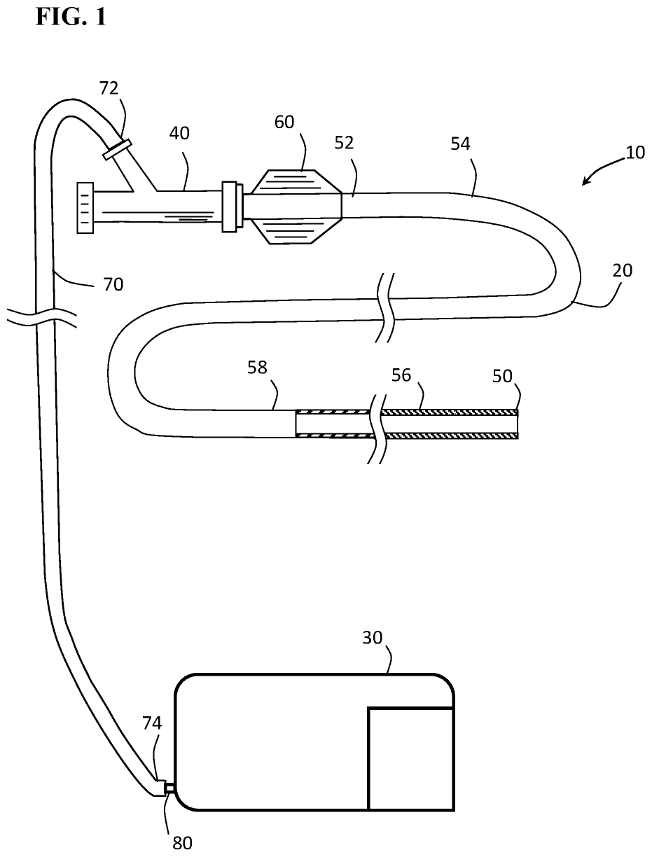

Methods and systems for capturing and removing an embolus or thrombus from an area of the body are herein described. While the terms “thrombectomy” and “thrombus” generally refer to removal of a specific type of embolus, the usage herein should be considered more broadly to include the removal additional types of emboli such as plaque, organized tissue fragments, clots and foreign objects that may block or restrict the normal flow of blood within the vasculature. In other nonvascular lumens within the body, the term “embolus” is herein construed more broadly, to include obstructions of a lumen such as “stones” lodged in a duct. FIG. 1 illustrates an embodiment of a thrombectomy system 10. Thrombectomy system 10 includes an elongate catheter 20, an aspiration pump 30 coupled together using connector 40. Catheter 20 has a distal end 50, a proximal end 52 and proximal, intermediate and distal sections (54, 56 and 58 respectively) positioned between said ends. Located at the proximal end of catheter 20 is a catheter hub 60 that facilitates the connection of connector 40 to the catheter. Connector 40 is also connected to aspiration pump 30 via tubing 70. Tubing 70 has a first end 72 connected to a port of connector 40 and a second end 74 which is connected to the intake 80 of pump 30.

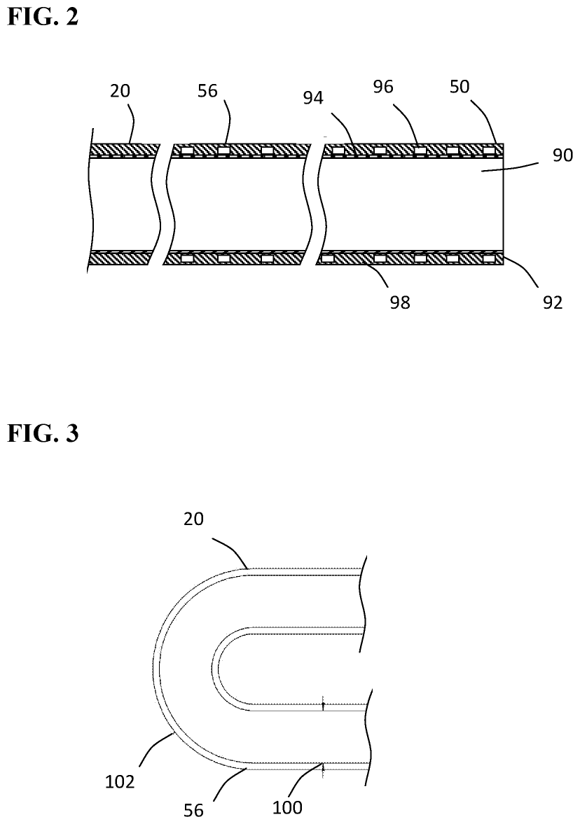

A partial cross sectional view of distal section 50 of catheter 20 is shown in FIG. 2. As shown, catheter 20 includes a lumen 90 defined by catheter wall 92. The catheter wall 92 at the distal section 50 may include an inner liner 94 surrounded by a helical reinforcement wire 96 and bonded to appropriate polymer 98. While not shown, the construction of catheter 20 may utilize known catheter technologies in the proximal and intermediate catheter sections that incorporate braiding and or coiling using metallic or non-metallic reinforcing filamentous materials to provide high strength while maintaining catheter flexibility. The incorporation of lubricious hydrophilic and or hydrophobic materials on the inner and or outer surface of the catheter is considered to be within the scope of known catheter construction techniques and suitable for use in a thrombectomy system according to embodiments.

With typical microcatheters used in the cerebrovasculature, the distal section of the catheter usually has an outer diameter (OD) of between 1 and 2 millimeters and an inner diameter (ID) of between 0.5 mm 1.5 mm with a wall thickness (WT) of about 0.25 mm which yields inner diameter to total wall thickness ratios of between 1.0 to 3.0. In this ratio range catheters generally have sufficient integrity to be navigated to a target site to perform their intended function. As this ratio decreases below 1.0 the catheters generally become too stiff and or the lumen size is too diminished to function for aspiration of thrombus. Similarly for ratio increases above 3.0 the catheters may become too flimsy to access a desired location or collapse under negative pressure (unless designed with sufficient high strength reinforcement). In a preferred embodiment the optimized catheter 20 has a wall thickness of about 0.25 mm and inner diameter greater than 2.0 mm yielding an ID to total wall thickness ratio greater than 4.0 while incorporating a helical reinforcement wire 96 as shown in FIG. 2. The helical reinforcement wire may be formed suitable biocompatible resilient materials including nitinol, stainless steel, titanium, cobalt chromium, carbon fiber, glass fiber and polymeric fibers like nylon, Kevlar or Spectra.

FIG. 3 shows a partial cross sectional view of distal section 56 of catheter 20 positioned in a bend. As previously discussed the preferred embodiment incorporates a helical reinforcement wire 96 which surrounds the inner diameter 100. With optimized construction the distal section 56 of catheter 20 can be positioned in a bend such that the outer diameter bend radius 102 is two times the inner diameter without kinking when the inner diameter is greater than 0.085 in.

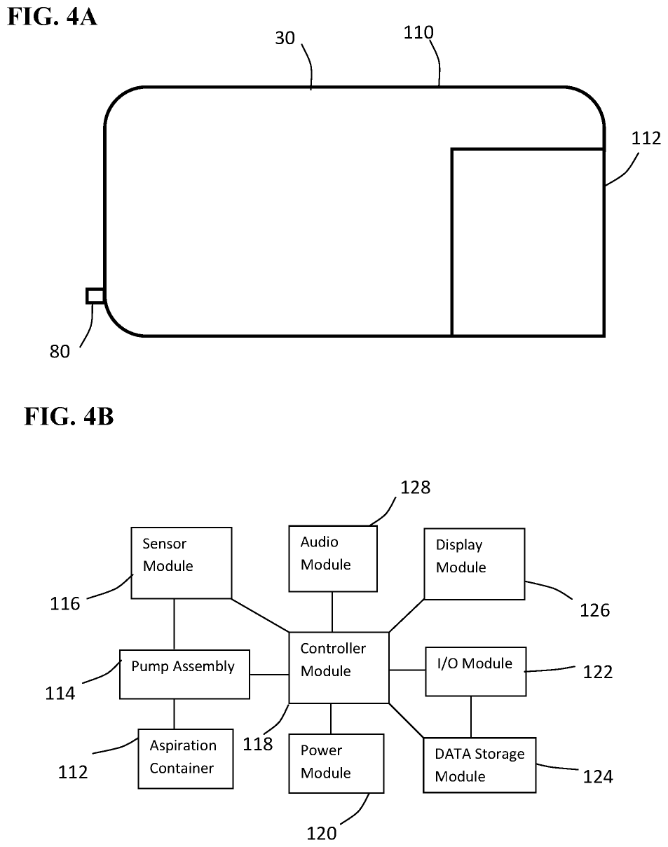

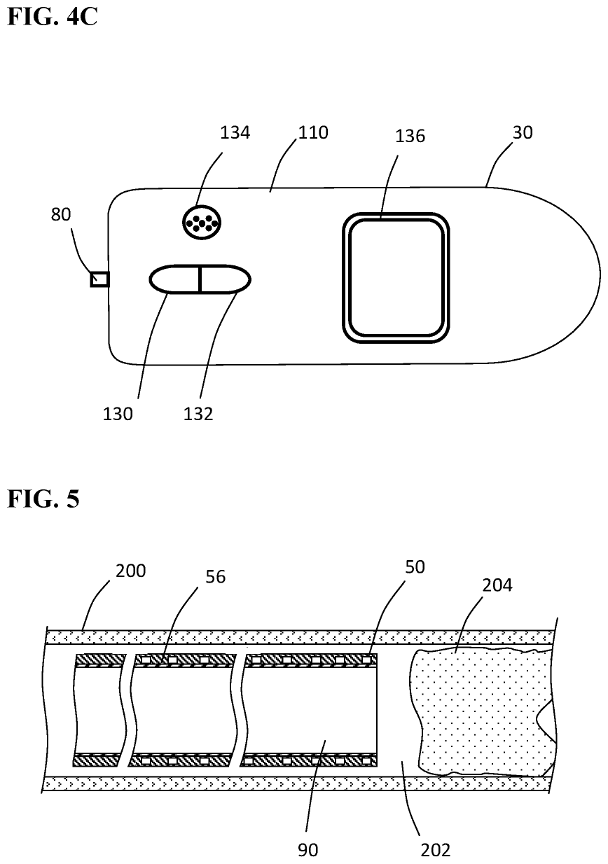

In addition to catheter 20, the thrombectomy system 10 includes aspiration pump 30 is shown in FIG. 4A. Aspiration pump 30 includes a housing 110 and a removable aspiration container 112. FIG. 4B illustrates the functional components of the pump contained within housing 110. The components include a pump assembly 114, a sensor module 116, a programmable controller module 118 and a power module 120. The aspiration pump includes other modules such as an input/output module 122 whereby the programmable controller can wirelessly receive or send programs or data to or from external devices, a data storage module 124, a display module 126 and an audio module 128. The controller module is capable of controlling the pump assembly 114 to provide negative pressure to the catheter and thus suction when removing thrombus. The controller also has the capability to alternate between negative and positive pressure to apply a dynamic load to thrombus to dislodge any thrombus that may become lodged in the distal end of the catheter. The pump has a limiting feature that does not apply positive pressure in such a way that thrombus or debris is expelled from the distal end of the catheter. The frequency of alternating between negative and positive pressures as well as the magnitude of the pressure can be wirelessly input to controller via the input/output module using known wireless protocols including Bluetooth and WIFI. Alternatively, the controller may be programmed manually using the lighted first and second push buttons 130 and 132 respectively, positioned on the top of housing 110 shown in FIG. 4C. Also located on housing 110, are speaker 134 and display 136. Speaker 134 is used to provide audible feedback to the user during programming of the controller and operation of the thrombectomy system. Display 136 provides visual feedback for the pressure settings as well as data from the sensor module monitoring the pressure. Display 136 is coupled to the programmable controller module and is preferably a Thin Film Transistor liquid crystal display with touch screen capability however other types of display screens may be suitable. Together the first and second push buttons 130 & 132, speaker 134 and display 136 form a user interface that allows for programming of the controller, display of data during pump operation and providing feedback for any alarms. For instance, an alarm may be set to trigger when negative pressure reaches a certain limit. When the alarm is triggered the user interface may provide feedback to the user in the form of alternating flashing of first and second push buttons, audible chirping through the speaker, flashing of the display or any combination thereof, informing the user to take some action.

As previously discussed small diameter microcatheters that have been used in the past to for thrombus removal have difficulty in removing thrombus partially due to the small catheter lumen requiring the piecemeal breakup of the thrombus into smaller pieces. Additionally these small diameter microcatheters have difficulties in removing thrombus through aspiration because the diameter of the catheter lumen in relation to the inner diameter of the vessel in which the thrombus is lodged is generally in the range of 30 to 40 percent. During aspiration, this difference in diameter allows blood positioned proximal to the distal end of the catheter to be drawn into the catheter reducing the amount of suction being applied directly to the thrombus. To compensate for the reduced suction force the catheter tip is typically positioned directly adjacent or in contact with the thrombus which can cause the catheter lumen to become plugged. FIG. 5 illustrates catheter 20 of thrombectomy system 10 located within a vessel 200. The distal section 56 of catheter 20 is situated within vessel lumen 202 whereas the catheter distal end 50 is positioned adjacent the thrombus 204. As shown in FIG. 5 the diameter of catheter lumen 90 is greater than 50 percent of the diameter of vessel lumen 202. When the catheter lumen to vessel lumen ratio is greater than 50 percent aspiration through the catheter lumen becomes more efficient the suction force from aspiration is directed more towards the thrombus as opposed to suctioning blood positioned proximal to the catheter distal end. This increased suction efficiency allows the thrombus to stretched and drawn into the catheter lumen typically without fragmentation.

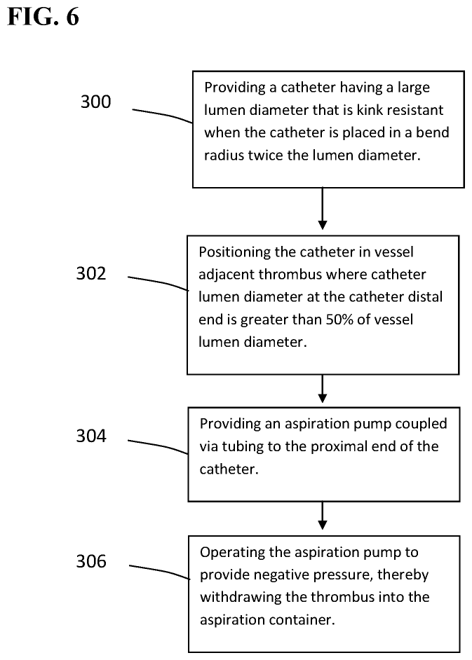

FIG. 6 shows a method of performing a thrombectomy procedure using thrombectomy system 10. The method comprises the steps of:
providing an elongate catheter having proximal and distal ends and an inner diameter greater than 0.085 in;
positioning the distal end of the catheter within a vessel lumen wherein the inner diameter of the catheter distal end is greater than 50% of the inner diameter of the vessel lumen and adjacent a thrombus;
providing an aspiration pump having an aspiration container;
coupling the aspiration pump to the catheter using a connector;
operating the aspiration pump to provide negative pressure to the lumen of the catheter thereby suctioning thrombus through the catheter lumen and into the aspiration container;
withdrawing the catheter from the vasculature.
   When operating thrombectomy system 10 according to the aforementioned method steps, thrombus can be efficiently and effectively removed from the vasculature.

Novel devices, systems and methods have been disclosed to perform thrombectomy procedures within the vessel of a mammal. Although preferred embodiments have been described, it should be understood that various modifications including the substitution of elements or components which perform substantially the same function in the same way to achieve substantially the same result may be made by those skilled in the art without departing from the scope of the claims which follow.

What is claimed is:

1. A thrombus removal system for use in the neurovasculature comprising:
an elongate tubular catheter having proximal and distal segments, a wall, an inner diameter, an outer diameter, proximal and distal ends and a lumen extending there between, said catheter distal segment having reinforcement to provide kink resistance such that when outer diameter has a bend radius of at least two times the inner diameter the catheter does not kink and said catheter distal segment having a sufficient radial resistance such that the catheter distal segment does not collapse when subjected to a negative pressure; and
an aspiration pump having a pump assembly including a length tubing having a connector end, a controller, an aspiration container, a housing, a user interface, and an operable mode, said connector end being adapted to couple to the proximal end of said catheter wherein when said aspiration pump is coupled to said catheter positioned within the vasculature and operated, said pump can controllably cycle between supplying negative and positive pressure to said catheter to aspirate a thrombus through the catheter to the pump aspiration container.

2. A thrombus removal system as in claim 1 wherein said catheter has an inner diameter greater than 0.085 inches.

3. A thrombus removal system as in claim 1 wherein said catheter wall has a thickness and the ratio of the inner diameter to the diametric wall thickness is greater than 4.

4. A thrombus removal system as in claim 1 wherein said positive pressure does propel aspirated material within the catheter distal to the catheter distal end.

5. A thrombus removal system as in claim 1 wherein said catheter remains functional when subjected to a negative pressure of 29 in Hg.

6. A thrombus removal system as in claim 1 wherein said aspiration pump user interface includes a data display.

7. A thrombus removal system as in claim 1 wherein said aspiration pump user interface includes audible feedback.

8. A thrombus removal system as in claim 1 wherein said aspiration pump user interface includes wireless transmission and or receiving of data.

9. A thrombus removal system as in claim 2 wherein said positive pressure does propel aspirated material within the catheter distal to the catheter distal end.

10. A thrombus removal system as in claim 2 wherein said catheter remains functional when subjected to a negative pressure of 29 in Hg.

11. A thrombus removal system as in claim 2 wherein said aspiration pump user interface includes a data display.

12. A thrombus removal system as in claim 2 wherein said aspiration pump user interface includes audible feedback.

13. A thrombus removal system as in claim 2 wherein said aspiration pump user interface includes wireless transmission and or receiving of data.

14. A method of removing thrombus from a vessel using a thrombus removal system comprising:
providing an elongate catheter having proximal and distal ends and an inner diameter greater than 0.085 in;
positioning the distal end of the catheter within a vessel lumen adjacent a thrombus wherein the inner diameter of the catheter distal end is greater than 50% of the inner diameter of the vessel lumen;
providing an aspiration pump having an aspiration container;
coupling the aspiration pump to the catheter using a connector;
operating the aspiration pump to provide negative pressure to the lumen of the catheter thereby suctioning thrombus through the catheter lumen and into the aspiration container.

15. A method of removing thrombus according to claim 14 wherein operating the aspiration pump includes providing cyclic negative and positive pressure.
